(19) United States
(12) Patent Application Publication
(10) Pub. No.: US 2008/0102092 A1
(43) Pub. Date: May 1, 2008

(54) ANTI-TRICHOPHYTON FILM FORMING AGENT AND REMEDIES FOR DERMATOPHYTOSIS

(76) Inventor: KOJI SASAYA, Yokohama-shi (JP)

Correspondence Address: J.C. Patents, Suite 250, 4 Venture, Irvine, CA, 92618, US

(21) Appl. No.: 11/959,303

(22) Filed: Dec. 18, 2007

Related U.S. Application Data
(63) Continuation-in-part of application No. 10/450,286, filed on Oct. 6, 2003, now abandoned.

(30) Foreign Application Priority Data
Dec. 11, 2000 (JP) 2000-376219

(51) Int. Cl. A61K 9/00; A61K 33/10; A61P 43/00

(52) U.S. Cl. 424/400; 424/687

(57) ABSTRACT

An anti-Trichophyton film containing calcium hydrogen carbonate and an anti-Trichophyton forming agent enable easy formation, on a human skin, of an antibacterial film containing calcium hydrogen carbonate while assigning antibacterial effect possessed by a natural material thereto by using scallop shell as a raw material, and form an antibacterial film of calcium hydrogen carbonate with the antibacterial effect of the natural material added thereto through a simple structure, thereby enhancing the curative effect to athlete's foot. The anti-Trichophyton film forming agent is obtained by mixing a powder obtained by calcinating scallop shell mainly composed of calcium carbonate with distilled water. Remedies for dermatophytosis are prepared by grinding and calcinating scallop shells having a crystalline structure due to the calcite structure of calcium carbonate.

### CROSS-REFERENCE TO RELATED APPLICATION

This application is a continuation-in-part application, and claims priority benefit, of application Ser. No. 10/450,286 having 35 U.S.C. 371(c) date of Oct. 6, 2003, which is a national phase application of international application No. PCT/JP2001/10552 filed on Dec. 3, 2001, which claims the priority benefit of Japan application No. 2000-376219 filed on Dec. 11, 2000.

### BACKGROUND OF THE INVENTION

1. Field of the Invention

The present invention relates to a Trichophyton-resistant film and an agent for forming the same. This invention relates to remedies for dermatophytosis.

2. Description of the Related Art

Various remedies for dermatophytosis have been provided for a long-term use for effectively preventing infection from other persons and to others persons. However, since these remedies contain a lot of chemically synthesized substances, adverse side effects to human bodies due to a long-term use thereof have been worried.

For solving the problem above, the inventors of this invention have found that a powder obtained by calcining and pulverizing scallop shells, which have been disposed of as wastes, can be utilized as a natural product having an anti-fungal activity without adverse effects on the human body. Accordingly, the object of this invention taking the above situation into consideration is to obtain a raw material for the remedies of dermatophytosis from the scallop shell, and to provide remedies of dermatophytosis that are harmless to the human body by taking advantage of natural products while attempting to effectively utilizing waste of the natural products.

Meanwhile a newspaper dated Jan. 12, 2006 reported that an anti-Trichophyton agent is produced by dissolving a baked scallop shell powder into distilled water. That agent is directly applied to the affected part of the patient. Calcium hydrogen carbonate (Ca(HCO₃)₂) contained in the shell prevents proliferation of Trichophyton fungus and causes the extinction of the same.

However because calcium hydrogen carbonate (Ca(HCO₃)₂) is very unstable, its effect for athlete's foot remedies is insufficient.

Hereupon Trichophyton is included in pathogenic fungi of Dermatophytosis such as an athlete's foot syndrome.

### SUMMARY OF THE INVENTION

The present invention provides a stable anti-Trichophyton film containing calcium hydrogen carbonate and its forming agent for forming an anti-Trichophyton film including calcium hydrogen carbonate, the agent being a mixture of a powder obtained by calcinating powdered scallop shell mainly composed of calcium carbonate at a temperature of about 900° C. or higher and distilled water.

According to the present invention, because the agent is obtained by mixing the calcium oxide powder obtained by baking scallop shell mainly composed of calcium carbonate at a temperature of about 900° C. or higher with distilled water, it has an antibacterial component as a natural product, and shows alkaline property in a state where the calcium oxide obtained by calcination of scallop shell powder is dissolved in the distilled water to transform to calcium hydroxide. Therefore, inhabitation of Trichophyton can be suppressed by applying it to a skin such as an athlete's foot affected part because the anti-Trichophyton film including calcium hydrogen carbonate becomes more stable and sustainable under concentrated water vapor and carbon dioxide gas transpired from the patient's living skin.

Further, the calcium oxide powder shows alkaline property as an aqueous solution of calcium hydroxide by the reaction shown by CaO+H₂O→Ca(OH)₂, and exhibits antibacterial effect by the alkaline property.

That is, further, when this anti-Trichophyton film forming agent is applied onto the skin, it is laid in a thin film state where concentrated carbon dioxide gas transpiring from the skin itself are extremely easy to be absorbed, and the dissolution of the carbon dioxide or the like is continued, whereby the reaction represented by the following expressions is continued to form a film including calcium hydrogen carbonate.
Ca(OH)₂+CO₂→CaCO₃+H₂O
CaCO₃+H₂O+CO₂→Ca(HCO₃)₂

With a film comprising the above anti-Trichophyton film forming agent under a normal atmosphere, formation of calcium hydrogen carbonate is insufficient for quantity of the formed film and stability of the obtained film because of both low water vapor and carbon dioxide partial pressures under such normal atmosphere.

This film including calcium hydrogen carbonate also has antibacterial effect and sterilization effect to Trichophyton in itself as described above, so an antibacterial film also having the antibacterial component possessed by the scallop shell as the natural product can be easily obtained. Further the calcium hydrogen carbonate film containing water and carbon dioxide is a little soft while dried calcium carbonate or calcium hydroxide film is hard and stiff so that the calcium carbonate or calcium hydroxide film is not formed on a skin which is soft.

The invention also provides a remedy for dermatophytosis as a product obtained by pulverizing shells having a calcite crystal structure of calcium carbonate.

The above-mentioned remedy of this invention consists of the powdered shell having a calcite crystal structure of calcium carbonate, and it may be a mixture of calcium carbonate powder of porous particles and a calcium oxide powder prepared by calcining the calcium carbonate, and preferably it has good effect when it is dissolved in water.

### DESCRIPTION OF EMBODIMENTS

The present inventors found that a film composed of calcium hydrogen carbonate can effectively act as an anti-Trichophyton film, when formed on a human skin that is an athlete's foot affected part so as to cover the affected part, to suppress inhabitation of Trichophyton thereon.

To explain this point, it is known that Trichophyton grows on human skin, the skin tissue of which includes an upper layer part formed of keratin by taking the keratin as a nutrient, and also it is confirmed by the present invention that inhabitation of Trichophyton can be suppressed by forming an antibacterial film composed of calcium hydrogen carbonate (Ca(HCO₃)₂) so as to cover an inhabiting portion of Trichophyton. It is also confirmed that when Trichophyton is disposed on the antibacterial film composed of calcium hydrogen carbonate, the presence of the film disables the Trichophyton fungus disposed on the antibacterial film from directly taking the keratin, and the Trichophyton cannot survive.

Further, because calcium hydrogen carbonate in a film composed of calcium hydrogen carbonate is stably present in a wetted state thereof, calcium hydrogen carbonate in the anti-Trichophyton film absorbs water (H₂O) and carbon dioxide (CO₂). Moisture (liquid) always transpires from the skin including the affected part as sweat or the like, and the moisture is actively taken into the anti-Trichophyton film also in the affected part which is apt to be wetted, and the form of the film is thereby maintained. Since the moisture transpiring from the skin is thus taken into the anti-Trichophyton film, the affected part is apt to be kept in a dry state as the whole, and it is supposed that the suppression of inhabitation of Trichophyton can be thereby also sustainable.

Since the antibacterial film by calcium hydrogen carbonate can suppress the inhabitation of Trichophyton with excellent sterilization effect and antibacterial effect, the present invention has an object to enable formation, on a human skin, of an antibacterial film by calcium hydrogen carbonate while assigning antibacterial effect possessed by a natural material thereto by using the scallop shell as a raw material, and easily form an antibacterial film of calcium hydrogen carbonate with the antibacterial effect of the natural material added thereto through a simple structure, thereby enhancing the curative effect to athlete's foot.

The anti-Trichophyton film forming agent of the present invention is obtained by mixing two materials such as a calcium oxide powder obtained by calcinating scallop shell mainly composed of calcium carbonate at a temperature of about 900° C. or higher and distilled water. Such calcinated powder is dissolved in the distilled water by being mixed therewith. This powder is obtained, as described above, by pulverizing scallop shell dried with sun exposure, for example, to a particle diameter of about 1 to about 500 μm, for example 200 μm, to prepare calcium carbonate powder composed of scallop shell powder, calcinating the calcium carbonate powder at a temperature at which it is changed to calcium oxide to prepare calcium oxide powder composed of scallop shell powder, and mixing the calcium carbonate powder and the calcium oxide powder which are both composed of scallop shell powder, and this powder has antibacterial effect and sterilization effect because it includes the antibacterial component derived from the scallop shell as the natural product and shows alkaline property. The powder is mixed with distilled water preliminarily free from gas, and laid in the form of an aqueous solution of calcium hydroxide while retaining the antibacterial component.

This anti-Trichophyton film forming agent solution is filled in, for example, a spray container and applied as an aqueous solution onto an athlete's foot affected part by spraying, whereby the athlete's affected part is covered with a thin membrane-like liquid film. And then, the above-mentioned calcium hydrogen carbonate-forming reaction is promoted on the skin by natural dissolution of carbon dioxide into the film under concentrated water vapor and carbon dioxide gas. Therefore, an anti-Trichophyton film having the antibacterial component and composed of calcium hydrogen carbonate can be easily obtained without efforts with applied on the living human skin.

The remedy for dermatophytosis of the present invention uses shells having at least a calcite crystal structure of calcium carbonate as a main ingredient thereof. Scallop shells are one of the representative examples of such shells, which are different from other shells in that most part of the scallop shells comprises this crystal structure. In the ecology of the scallops, they are featured in that they can quickly move for escaping from being captured by their predators (such as starfishes), as expressed as “swimming in the sea” by jetting sea water out of the shell by opening and shutting the shell. Shell adductors are well developed for enabling this action, and the shell itself is relatively thin and lightweight while having high mechanical strength. The shell comprises a calcite crystal structure of calcium carbonate aligned as a leaf-like texture at the inner face side of the shell. The calcite crystal structure of calcium carbonate has a plate-like texture in the inner layer of the shell (a core layer in the direction of thickness of the shell). Since the main part of the shell structure (the portion except the surface layer and hinge) is composed of the inner face side and inner layer of the shell, the shell can be thin and lightweight while having a high mechanical strength.

As described above with respect to the main part of the scallop shell, the inner face side has the leaf-like texture of the calcite crystal structure of calcium carbonate (needle-like crystals are aligned by being compacted as Kenzan (a needle-type frog), while the inner layer of the shell has a plate-like structure of the calcite crystal structure of calcium carbonate (many layers in which the needle-like crystals are aligned in one direction are stacked like a plywood, and the needle-like crystals are oriented in different directions for every layers). Accordingly, as described later, the powder obtained by pulverizing the scallop shells is porous and maintains calcite structure. On the other hand, while the calcium carbonate crystals are stacked by being spread on a plane in other kind of shells, and the shell has pearl luster, the granules prepared from such shells does not become porous.

As described above, the scallop shells are used as the raw material for obtaining the remedy of dermatophytosis, and though the shells have been disposed of as useless after removing the adductor of the shell, useful utilization of such waste is possible. The waste shells are collected, and dried in the sunlight for hardening. Then, the shells thus hardened by drying in the sunlight are pulverized to a granule of a diameter of about 200 μm. The pulverizing method is not particularly restricted, and pulverizers known in the art may be used. The porous granules of calcium carbonate are obtained as described above.

Subsequently, a part of the calcium carbonate powder comprising porous particles are used for obtaining a powder of calcium oxide by heating the particles at about 1000° C. for several minutes to several hours in a ceramic pot. A product is obtained by mixing the calcium oxide powder and calcium carbonate powder, and a remedy for dermatophytosis is prepared by dispersing the product in water.

Since the calcium carbonate powder in the remedy for dermatophytosis is the powder of scallop shell that is a natural product, as described above, it has an antimicrobial activity inherent to the natural products. The natural product is used herein as a contrast to chemically synthesized product, and cultivated scallop shells also belong to the natural product. The calcium oxide powder also exhibits a high antimicrobial activity due to its alkaline property. Accordingly, the remedy of the invention exhibits an antimicrobial activity inherent in the natural products and an antimicrobial activity due to alkaline property, and has properties that are evaded by microorganisms such as Tricophyton that causes dermatophytosis. Proliferation as well as survival of the microorganisms are inhibited at the lesion, and epidermis afflicted with dermatophytosis is restored by the human body's natural healing power.

The product is prepared by mixing the shell powder, which comprises porous particles and is the calcite crystal structure having the calcium carbonate powder, and the calcium oxide powder prepared by calcining the calcium carbonate powder, and by dispersing the mixture in water in the example above. However, the remedy of the invention is not limited thereto. For example, the product may comprise only the calcium carbonate powder or may comprise only the calcium oxide powder. Further, the product itself may be used without dispersing in water.

### INDUSTRIAL APPLICABILITY

According to the present invention as described above, the remedy for dermatophytosis exhibits the antimicrobial activity inherent to the natural product and an alkaline property. Accordingly, the human body is not adversely affected by applying the remedy while inhibiting viability of microorganisms that is considered to cause the remedy of dermatophytosis to enable the natural healing power of the human body to be effectively manifested. It is another advantage that no adverse effect is observed after a long-term use since the product comprises natural materials without containing any chemically synthesized substances.

Use of the shell as the raw material permits the shell that has been mostly disposed of as wastes to be effectively used, thereby greatly contributing to reduce the amount of industrial wastes. The product of the invention can exert an excellent practical effect by providing the product at a low cost, since the remedy having a simple constitution is produced without using any complex process.

1. An anti-Trichophyton film including calcium hydrogen carbonate and water, wherein the agent being disposed on the human skin of a patient under concentrated water vapor and carbon dioxide transpired from said skin.

2. An anti-Trichophyton film according to claim 1, wherein said calcium hydrogen carbonate is obtained by calcinating scallop shell mainly composed of calcium carbonate at a temperature of about 900° C. or higher and dissolving the obtained calcium oxide into water to convert calcium oxide to calcium hydroxide.

3. An anti-Trichophyton film forming agent for forming an anti-Trichophyton film including calcium hydrogen carbonate, wherein the agent being composed of a mixture of a powder obtained by calcinating scallop shell mainly composed of calcium carbonate at a temperature of about 900° C. or higher and distilled water.

4. An anti-dermatophyte agent being harmless to lesions of a human body and consisting of (a) a calcined product, containing a major portion of calcium oxide and prepared by pulverizing and calcining scallop shells and (b) a pulverized product, containing a major portion of calcium carbonate and having high porosity, prepared by pulverizing uncalcined scallop shells.

5. An anti-dermatophyte agent according to claim 4, wherein the pulverized products have an average particle size of at least about 200 micron meter.

6. An anti-dermatophyte agent according to claim 4, wherein the calcined product is prepared with calcining at least at about 1000 degree. C.

7. An anti-dermatophyte agent according to claim 4, wherein said agent is in an aqueous solution or in an aqueous dispersion.

8. A method for suppressing the growth of dermatophyte in vivo being harmless to lesions of a human body and comprising:
administrating an anti-dermatophyte agent consisting of (a) a calcined product, containing a major portion of calcium oxide and prepared by pulverizing and calcining scallop shells and (b) a pulverized product, containing a major portion of calcium carbonate, and having high porosity prepared by pulverizing scallop shells.

9. The method of claim 8, wherein said administrating is by administrating an aqueous solution or dispersion of the anti-dermatophyte agent.
